SEQUENCE LISTING (21 sequences)

SEQ ID NO: 1 - protein, 502 aa, Escherichia coli
MTEKKYIVALDQGTTSSRAVVMDHDANIISVSQREFEQIYPKPGWVEHDPMEIWATQSSTLVEVLAKADISSDQIAAIGITNQRETTIVWEKETGKPIYNAIVWQCRRTAEICEHLKRDGLEDYIRSNTGLVIDPYFSGTKVKWILDHVEGSRERARRGELLFGTVDTWLIWKMTQGRVHVTDYTNASRTMLFNIHTLDWDDKMLEVLDIPREMLPEVRRSSEVYGQTNIGGKGGTRIPISGIAGDQQAALFGQLCVKEGMAKNTYGTGCFMLMNTGEKAVKSENGLLTTIACGPTGEVNYALEGAVFMAGASIQWLRDEMKLINDAYDSEYFATKVQNTNGVYVVPAFTGLGAPYWDPYARGAIFGLTRGVNANHIIRATLESIAYQTRDVLEAMQADSGIRLHALRVDGGAVANNFLMQFQSDILGTRVERPEVREVTALGAAYLAGLAVGFWQNLDELQEKAVIEREFRPGIETTERNYRYAGWKKAVKRAMAWEEHDE

SEQ ID NO: 2 - DNA, 1509 bp, Escherichia coli
  source 1..1509: mol_type = genomic DNA
atgactgaaaaaaaatatatcgttgcgctcgaccagggcaccaccagctcccgcgcggtcgtaatggatcacgatgccaatatcattagcgtgtcgcagcgcgaatttgagcaaatctacccaaaaccaggttgggtagaacacgacccaatggaaatctgggccacccaaagctccacgctggtagaagtgctggcgaaagccgatatcagttccgatcaaattgcagctatcggtattacgaaccagcgtgaaaccactattgtctgggaaaaagaaaccggcaagcctatctataacgccattgtctggcagtgccgtcgtaccgcagaaatctgcgagcatttaaaacgtgacggtttagaagattatatccgcagcaataccggtctggtgattgacccgtacttttctggcaccaaagtgaagtggatcctcgaccatgtggaaggctctcgcgagcgtgcacgtcgtggtgaattgctgtttggtacggttgatacgtggcttatctggaaaatgactcagggccgtgtccatgtgaccgattacaccaacgcctctcgtaccatgttgttcaacatccataccctggactgggacgacaaaatgctggaagtgctggatattccgcgcgagatgctgccagaagtgcgtcgttcttccgaagtatacggtcagactaacattggcggcaaaggcggcacgcgtattccaatctccgggatcgccggtgaccagcaggccgcgctgtttggtcagttgtgcgtgaaagaagggatggcgaagaacacctatggcactggctgctttatgctgatgaacactggcgagaaagcggtgaaatcagaaaacggcctgctgaccaccatcgcctgcggcccgactggcgaagtgaactatgcgttggaaggtgcggtgtttatggcaggcgcatccattcagtggctgcgcgatgaaatgaagttgattaacgacgcctacgattccgaatatttcgccaccaaagtgcaaaacaccaatggtgtgtatgtggttccggcatttaccgggctgggtgcgccgtactgggacccgtatgcgcgcggggcgattttcggtctgactcgtggggtgaacgctaaccacattatacgcgcgacgctggagtctattgcttatcagacgcgtgacgtgctggaagcgatgcaggccgactctggtatccgtctgcacgccctgcgcgtggatggtggcgcagtagcaaacaatttcctgatgcagttccagtccgatattctcggcacccgcgttgagcgcccggaagtgcgcgaagtcaccgcattgggtgcggcctatctcgcaggcctggcggttggcttctggcagaacctcgacgagctgcaagagaaagcggtgattgagcgcgagttccgtccaggcatcgaaaccactgagcgtaattaccgttacgcaggctggaaaaaagcggttaaacgcgcgatggcgtgggaagaacacgacgaataa

SEQ ID NO: 3 - DNA, 25 bp, synthetic construct
  source 1..25: mol_type = other DNA
atggtgtgagtggacgttaaaatgc

SEQ ID NO: 4 - DNA, 53 bp, synthetic construct
  source 1..53: mol_type = other DNA
ggcgtttgccacctgtgcaatattacataatgtacatccgtagttaactttcc

SEQ ID NO: 5 - DNA, 56 bp, synthetic construct
  source 1..56: mol_type = other DNA
ggaaagttaactacggatgtacattatgtaatattgcacaggtggcaaacgccacc

SEQ ID NO: 6 - DNA, 26 bp, synthetic construct
  source 1..26: mol_type = other DNA
gagattcgcgatctgttcagtaaacg

SEQ ID NO: 7 - DNA, 90 bp, synthetic construct
  source 1..90: mol_type = other DNA
gtagaacacgacccaatggaaatctggacgacccaaagctccacgctggtagaagtgctggcgaaagccgatatcagttccgatcaaatt

SEQ ID NO: 8 - DNA, 19 bp, synthetic construct
  source 1..19: mol_type = other DNA
cccaatggaaatctgggcc

SEQ ID NO: 9 - DNA, 22 bp, synthetic construct
  source 1..22: mol_type = other DNA
cgacccaatggaaatctggacg

SEQ ID NO: 10 - DNA, 29 bp, synthetic construct
  source 1..29: mol_type = other DNA
gccaatgttagtctgaccgtatacttcgg

SEQ ID NO: 11 - DNA, 26 bp, synthetic construct
  source 1..26: mol_type = other DNA
gcttcgctgtaatatgactacgggac

SEQ ID NO: 12 - DNA, 88 bp, synthetic construct
  source 1..88: mol_type = other DNA
gatgagctgggtggaacggcatcaaccgttactggaacaacacgtactgtaacgcaacaggcactaccggtaacttaatttcccgcgc

SEQ ID NO: 13 - DNA, 27 bp, synthetic construct
  source 1..27: mol_type = other DNA
cgttactggaacaacacgtactgtaac

SEQ ID NO: 14 - DNA, 26 bp, synthetic construct
  source 1..26: mol_type = other DNA
cgttactggaacaacacgtactgtat

SEQ ID NO: 15 - DNA, 21 bp, synthetic construct
  source 1..21: mol_type = other DNA
ccaccggttgaggatttcccg

SEQ ID NO: 16 - DNA, 20 bp, synthetic construct
  source 1..20: mol_type = other DNA
cctgtgcgcaggctttttcg

SEQ ID NO: 17 - DNA, 87 bp, synthetic construct
  source 1..87: mol_type = other DNA
ggcgatcccggagattggaatacgcgtgccgcctttgccatcaatgttagtctgaccgtatacttcggaagaacgacgcacttctgg

SEQ ID NO: 18 - DNA, 28 bp, synthetic construct
  source 1..28: mol_type = other DNA
cgaagtatacggtcagactaacattgat

SEQ ID NO: 19 - DNA, 25 bp, synthetic construct
  source 1..25: mol_type = other DNA
agtatacggtcagactaacattggc

SEQ ID NO: 20 - DNA, 21 bp, synthetic construct
  source 1..21: mol_type = other DNA
gcgtgcagacggataccagag

SEQ ID NO: 21 - DNA, 25 bp, synthetic construct
  source 1..25: mol_type = other DNA
gctgtttggtacggttgatacgtgg

(19) United States
(12) Patent Application Publication
(10) Pub. No.: US 2026/0055418 A1
(43) Pub. Date: Feb. 26, 2026

(54) Fermentative Production of Oligosaccharides by Microbial Cells Utilizing Glycerol

(71) Applicant: Chr. Hansen A/S, Hoersholm (DK)

(72) Inventors: André Luis RODRIGUES, Rheinbreitbach (DE); Christian TROETSCHEL, Rheinbreitbach (DE); Henning FRERIGMANN, Rheinbreitbach (DE); Markus ENGLERT, Rheinbreitbach (DE); Elisabeth STAUDT-GRUNDMANN, Rheinbreitbach (DE)

(73) Assignee: Chr. Hansen A/S, Hoersholm (DK)

(21) Appl. No.: 19/104,688

(22) PCT Filed: Aug. 28, 2023
(86) PCT No.: PCT/EP2023/073564

§ 371 Date: Feb. 18, 2025

(30) Foreign Application Priority Data
Aug. 29, 2022 (EP) 22192613.2

(51) Int. Cl. C12N 15/70; C12N 9/10; C12N 9/12; C12N 9/88; C12P 19/18; C12R 1/19

(52) U.S. Cl. CPC C12N 15/70; C12N 9/1048; C12N 9/1205; C12N 9/88; C12P 19/18; C12R 2001/19; C12Y 204/01065; C12Y 207/0103; C12Y 402/03003

(57) ABSTRACT

Disclosed are methylglyoxal resistant genetically engineered microbial cells for the fermentative production of an oligosaccharide of interest, and methods for fermentative production of an oligosaccharide of interest using the methylglyoxal resistant microbial cells.

The present invention relates to the fermentative production of an oligosaccharide of interest by a microbial cell. More specifically, the invention relates to the fermentative production of an oligosaccharide of interest by a microbial cell which utilizes glycerol as carbon source and energy source.

### BACKGROUND

Amongst the group of oligosaccharides, Human Milk Oligosaccharides (HMOs) gained a lot of interest in recent years due to their health beneficial effects. For example, HMOs can prevent the attachment of pathogenic microorganisms to mucosal cells of the gastrointestinal tract, thereby preventing the emergence of diseases. In addition, HMOs exert prebiotic effects, i.e. they promote proliferation of non-pathogenic enteral bacteria. Beneficial effects on the maturing immune system and the nervous system were described too.

Due to the beneficial effects of HMOs, their industrial production became of particular interest as well. The economically most viable manufacturing of HMOs appears to be the fermentative production by genetically engineered microbial cells such as Escherichia coli cells, Bacillus subtilis cells, Corynebacterium glutamicum cells or Saccharomyces cerevisiae cells.

In general, industrial scale bioprocesses-such as the fermentative production of an oligosaccharide by/in a microbial cell-involve a production phase during which biosynthesis of the oligosaccharide of interest occurs. The production phase is followed by a product recovery phase wherein the oligosaccharide of interest is recovered from the culture broth and/or from the microbial cells. It is known that different substrates such as glucose, sucrose or glycerol can be used as carbon and energy source for the microbial cell in the production phase.

Glycerol is a carbon source for microbial cells that recently become economically attractive for fermentative production processes. An increase of glycerol supply in the markets occurred concomitant with the increase in production of biodiesel as an alternative to petrol-based fuels as glycerol is a byproduct of biodiesel production.

A microbial cell that gained particular interest in the fermentative production of oligosaccharides, in particular of human milk oligosaccharides, is the gram-negative bacterium Escherichia coli. Escherichia coli is a microorganism that is able to metabolize glycerol as carbon and energy source. The catabolic metabolism in E. coli can occur aerobically or anaerobically. The catabolic pathways of glycerol in E. coli are shown schematically in FIG. 1. The major aerobic pathway involves internalization of glycerol by a glycerol uptake facilitator “GlpF” (encoded by a glpF gene), a glycerol kinase (encoded by a glpK gene), and a glycerol-3-phosphate dehydrogenase (encoded by a glpD gene). The catabolic pathway for glycerol that is employed by E. coli in absence of oxygen requires the glycerol-3-phosphate dehydrogenase (encoded by the glpABC genes) instead of the glycerol-3-phosphate dehydrogenase in the conversion of glycerol-3-phosphate to dihydroxyacetone phosphate. An alternative anaerobic pathway for glycerol comprises a glycerol dehydrogenase (encoded by a gldA gene) and a dihydroxyacetone kinase (encoded by the dhaKLM genes) after glycerol uptake by the glycerol uptake facilitator. In all these metabolic pathways, dihydroxyacetone phosphate is generated as key metabolite which enters the central metabolism via the Embden-Meyerhof-Parnas (EMP) pathway.

Due to the location of the gldA gene in the genome of E. coli, i.e. close to a ptsA gene encoding the multiphosphoryl transfer protein 2 and to fsaB encoding a fructose-6-phosphate aldolase, and due to the higher affinity of the glycerol dehydrogenase GldA for dihydroxyacetone than for glycerol, it was suggested that the primary role of GldA is the conversion of dihydroxyacetone to glycerol in order to prevent E. coli cell from dihydroxyacetone toxicity. Moreover, dihydroxyacetone can also be spontaneously converted to methylglyoxal, a very toxic metabolite for E. coli that inhibits its growth at a concentration of as little as 0.3 mM.

Therefore, it is desired to render microbial cells for producing an oligosaccharide of interest less vulnerable to the toxicity of metabolites from the catabolic pathway of glycerol when the microbial cells are cultivated in the presence of glycerol as carbon source and energy source.

### SUMMARY

It has surprisingly been found that E. coli cells that are able to synthesize a human milk oligosaccharide intracellularly possess an improved production of the human milk oligosaccharide when they possess certain variants of their glycerol kinase without being affected by methylglyoxal toxicity.

Thus, in a first aspect, provided is a genetically engineered microbial cell for the production of an oligosaccharide of interest, wherein the genetically engineered microbial cell is able to intracellularly synthesize the oligosaccharide of interest when cultivated in the presence of glycerol as sole carbon source. The microbial cell possesses a glycerol permease (also known as glycerol uptake facilitator) for internalization of exogenous glycerol. The microbial cell further possesses a functional variant of the native E. coli glycerol kinase GlpK, wherein the amino acid sequence of the native E. coli GlpK is set forth in SEQ ID NO: 1, wherein the functional variant possesses an amino acid residue comprising a non-ionized but polar acting side chain at the position of its amino acid sequence which corresponds to amino acid position 55 in the amino acid sequence of the native E. coli GlpK, and/or wherein said functional variant possesses an amino acid comprising an anionic side chain at the position of its amino acid sequence which corresponds to amino acid position 231 in the amino acid sequence of the native E. coli GlpK. The genetically engineered microbial cell further possesses a metabolic pathway for intracellular biosynthesis of a nucleotide-activated monosaccharide comprising a nucleotide moiety and a monosaccharide moiety, wherein said nucleotide-activated monosaccharide is a donor substrate for transfer of its monosaccharide moiety to an acceptor molecule. The genetically engineered microbial cell further possesses a glycosyltransferase for the transfer of the monosaccharide moiety from the nucleotide-activated monosaccharide to an acceptor molecule for the intracellular biosynthesis of the oligosaccharide of interest.

In a second aspect, provided is the use of a genetically engineered microbial cell according to the first aspect for producing an oligosaccharide of interest. Hence, the use comprises the use of a genetically engineered microbial cell, wherein the genetically engineered microbial cell is able to intracellularly synthesize the oligosaccharide of interest when cultivated in the presence of glycerol as sole carbon source, wherein the genetically engineered microbial cell is able to intracellularly synthesize the oligosaccharide of interest when cultivated in the presence of glycerol as sole carbon source. The microbial cell possesses a glycerol permease (also known as glycerol uptake facilitator) for internalization of exogenous glycerol. The microbial cell further possesses a functional variant of the native E. coli glycerol kinase GlpK, wherein the amino acid sequence of the native E. coli GlpK is set forth in SEQ ID NO: 1, wherein the functional variant possesses an amino acid residue comprising a non-ionized but polar acting side chain at the position of its amino acid sequence which corresponds to amino acid position 55 in the amino acid sequence of the native E. coli GlpK, and/or wherein said functional variant possesses an amino acid comprising an anionic side chain at the position of its amino acid sequence which corresponds to amino acid position 231 in the amino acid sequence of the native E. coli GlpK. The genetically engineered microbial cell further comprises a metabolic pathway for the intracellular biosynthesis of a nucleotide-activated monosaccharide as a donor substrate of the monosaccharide moiety, and the genetically engineered microbial cell further comprises a glycosyltransferase for the transfer of the monosaccharide moiety from the nucleotide-activated monosaccharide to an acceptor molecule.

In a third aspect, provided is a method for fermentative production of an oligosaccharide of interest, wherein the method comprises
providing a genetically engineered microbial cell according to the first aspect for the production of the oligosaccharide of interest, i.e. a genetically engineered microbial cell being able to intracellularly synthesize the oligosaccharide of interest when cultivated in the presence of glycerol as sole carbon source, wherein the genetically engineered bacterial comprises a glycerol permease and a functional variant of the native E. coli glycerol kinase GlpK, wherein the amino acid sequence of the native E. coli GlpK is set forth in SEQ ID NO: 1, wherein the functional variant possesses an amino acid residue comprising a non-ionized but polar acting side chain at the position of its amino acid sequence which corresponds to amino acid position 55 in the amino acid sequence of the native E. coli GlpK, and/or wherein said functional variant possesses an amino acid comprising an anionic side chain at the position of its amino acid sequence which corresponds to amino acid position 231 in the amino acid sequence of the native E. coli GlpK, a metabolic pathway for the intracellular biosynthesis of a nucleotide-activated monosaccharide as a donor substrate of its monosaccharide moiety, and a glycosyltransferase for the transfer of the monosaccharide moiety from the nucleotide-activated monosaccharide to an acceptor molecule;
culturing the genetically engineered microbial cell in a culture medium and at conditions that are permissive for the genetically engineered microbial cell to intracellularly synthesize the oligosaccharide of interest, wherein the culture medium contains glycerol as carbon source for the genetically engineered microbial cell; and
optionally, retrieving the oligosaccharide of interest from the microbial cell and/or the culture medium.

In a further aspect, provided is a method for alleviating the toxicity of methylglyoxal to a microbial cell when the microbial cell is cultivated in the presence of glycerol as carbon source, the method comprises transforming the microbial cell to possess a functional variant of the native E. coli glycerol kinase GlpK, wherein the amino acid sequence of the native E. coli GlpK is set forth in SEQ ID NO: 1, wherein the functional variant possesses an amino acid residue comprising a non-ionized but polar acting side chain at the position of its amino acid sequence which corresponds to amino acid position 55 in the amino acid sequence of the native E. coli GlpK, and/or wherein said functional variant possesses an amino acid comprising an anionic side chain at the position of its amino acid sequence which corresponds to amino acid position 231 in the amino acid sequence of the native E. coli GlpK.

In a further aspect, provided is a method for decreasing responsivity of a microbial cell to methylglyoxal toxicity during fermentative production of an oligosaccharide of interest by the microbial cell when the microbial cell is cultivated to produce the oligosaccharide of interest in the presence of exogenous glycerol as carbon source, the method comprises transforming the microbial cell to possess a functional variant of the native E. coli glycerol kinase GlpK, wherein the amino acid sequence of the native E. coli GlpK is set forth in SEQ ID NO: 1, wherein the functional variant possesses an amino acid residue comprising a non-ionized but polar acting side chain at the position of its amino acid sequence which corresponds to amino acid position 55 in the amino acid sequence of the native E. coli GlpK, and/or wherein said functional variant possesses an amino acid comprising an anionic side chain at the position of its amino acid sequence which corresponds to amino acid position 231 in the amino acid sequence of the native E. coli GlpK.

### BRIEF DESCRIPTION OF THE DRAWINGS

FIG. 1 illustrates the catabolic pathways of glycerol in E. coli.

FIG. 2 illustrates the nucleotide sequence of the protein-coding region of the native E. coli glpK gene, and the deduced amino acid sequence of the native E. coli glycerol kinase GlpK.

FIG. 3 shows graphs comparing the growth (A) and 3-FL production (B) of an E. coli strain producing 3-FL which strain contains the native E. coli (K12) GlpK enzyme (control) and a descendent of this control strain which contains the functional variant GlpK (A55T) instead of the native E. coli (K12) GlpK.

FIG. 4 displays graphs comparing the growth (A) and 2′-FL production (B) of an E. coli strain producing 2′-FL which strain contains the native E. coli GlpK enzyme (control), a descendent of this control strain which contains the functional variant GlpK (A55T) of the native E. coli GlpK instead of the native E. coli GlpK, and another descendent of the control strain which contains the functional variant GlpK (G231D) of the native E. coli GlpK instead of the native E. coli GlpK.

FIG. 5 shows graphs comparing the growth (A) and LNT production (B) of an E. coli strain producing LNT which strain contains the native E. coli GlpK enzyme (control) and a descendent of this control strain which contains the functional variant GlpK (A55T) of the native E. coli GlpK instead of the native E. coli GlpK.

FIG. 6 displays graphs which compare the growth profiles of E. coli strains producing 2′-FL (A) or LNT (B), wherein the growth profiles of these strains bearing their endogenous mgsA gene are compared to the growth profiles of respective descendent strains lacking their endogenous mgsA gene.

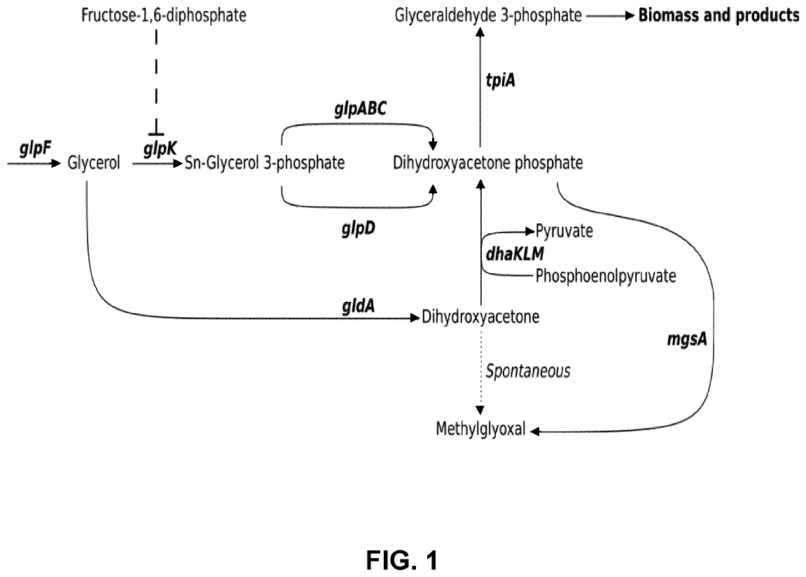

### DETAILED DESCRIPTION

According to the first aspect, provided is a genetically engineered microbial cell for the fermentative production of an oligosaccharide of interest.

The term “microbial cell” as used herein refers to unicellular organisms. The microbial cell can be a prokaryotic cell or can be a eukaryotic cell. Suitable procaryotic cells include bacterial cells and archaebacterial cells. Suitable eukaryotic cells include yeast cells and fungal cells.

In some embodiments, the prokaryotic cell is a bacterial cell. The bacterial cell is a bacterial cell of a genus selected from the group consisting of Bacillus, Bifidobacterium, Citrobacter, Clostridium, Corynebacterium, Enterococcus, Erwinia, Escherichia, Lactobacillus, Lactococcus, Micrococcus, Micromonospora, Pantotea, Pectobacterium, Proprionibacterium, Pseudomonas, Rhodococcus, Sporolactobacillus, Streptococcus and Xanthomonas. Suitable bacterial species within said genera are Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, Bacillus circulans, Bifidobacterium longum, Bifidobacterium infantis, Bifidobacterium bifidum, Citrobacter freundii, Clostridium cellulolyticum, Clostridium ljungdahlii, Clostridium autoethanogenum, Clostridium acetobutylicum, Corynebacterium glutamicum, Enterococcus faecium, Enterococcus thermophiles, Erwinia herbicola (Pantoea agglomerans), Escherichia coli, Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, Lactococcus lactis, Pantoea citrea, Pectobacterium carotovorum, Proprionibacterium freudenreichii, Pseudomonas fluorescens, Pseudomonas aeruginosa, Streptococcus thermophiles and Xanthomonas campestris. In another embodiment, the microbial cell is an Escherichia coli cell.

In some embodiments, the eukaryotic cell is a yeast cell. A suitable yeast cell can be selected from a genus selected from the group consisting of Saccharomyces sp., Saccharomycopsis sp., Pichia sp., Hanensula sp., Kluyveromyces sp., Yarrowia sp., Rhodotorula sp., and Schizosaccharomyces sp. Additionally and/or alternatively, the yeast cell is a Saccharomyces cerevisiae cell, a Pichia pastoris cell, or a Hanensula polymorpha cell.

The microbial cell is a microbial cell for producing an oligosaccharide of interest. The terms “for producing” as used herein with respect to the production of the oligosaccharide of interest means that the microbial cell is able to synthesize the oligosaccharide of interest intracellularly when being cultured in a medium and under conditions that are permissible for the microbial cell to intracellularly synthesize the oligosaccharide of interest.

The term “oligosaccharide” as used herein refers to a saccharide molecule consiting of at least three monosaccharide moieties, but of no more than twenty monosaccharide moieties, preferably of no more than 12 monosaccharide moieties, and more preferably of no more than 10 monosaccharide moieties, wherein each of said monosaccharide moieties is bound to at least one other of the monosaccharide moieties by a glycosidic linkage. The oligosaccharide of interest can consist of a linear chain of monosaccharide moieties or it can consist of a branched chain of monosaccharide moieties.

The monosaccharide moieties of the oligosaccharide of interest can be selected from the group consisting of aldoses (e.g. arabinose, xylose, ribose, desoxyribose, lyxose, glucose, idose, galactose, talose, allose, altrose, mannose), ketoses (e.g. ribulose, xylulose, fructose, sorbose, tagatose), deoxysugars (e.g. rhamnose, fucose, quinovose), deoxy-aminosugars (e.g. N-acetylglucosamine, N-acetyl-mannosamine, N-acetyl-galactosamine), uronic acids (e.g. galacturonsäure, glucuronsäure) and ketoaldonic acids (e.g. N-acetylneuraminic acid).

The term “oligosaccharide of interest” as used herein refers to the oligosaccharide that is supposed to be produced by the genetically engineered microbial cell. Typically, the microbial cell has been selected and/or genetically engineered to intracellularly synthesize a particular oligosaccharide, i.e. the oligosaccharide of interest. Any other oligosaccharide than the oligosaccharide of interest that may also be synthesized by the microbial cell is considered to be a by-product, regardless of whether the other oligosaccharide is a reaction product of an intermediate reaction in the biosynthesis of the oligosaccharide of interest, constitutes a product of another metabolic pathway than the metabolic pathway for the biosynthesis of the oligosaccharide of interest, or is an undesired by product due to the promiscuity of an enzyme involved in the metabolic pathway for synthesizing the oligosaccharide of interest.

Typically, the oligosaccharide of interest is not naturally occurring in the native cells of microbial species chosen for being genetically engineered to produce the oligosaccharide of interest. Thus, the genetically engineered microbial cell is a microbial cell that has been genetically engineered to be able to produce the oligosaccharide of interest.

In some embodiments, the oligosaccharide of interest is a human milk oligosaccharide (HMO), i.e. an oligosaccharide selected from the group of oligosaccharides that are present in human milk. Human milk oligosaccharides constitute a diverse mixture of oligosaccharides that are not digestible by a human. Human milk is unique with respect to its oligosaccharide composition and quantities. To date, more than 150 structurally distinct HMOs have been identified. The vast majority of HMOs are characterized by a lactose moiety (Gal-β1,4-Glc) at their reducing end. The HMOs contain at least one fucose moiety, at least one sialic acid moiety and/or at least one N-acetylglucosaminyl moiety. More generally speaking, the monosaccharides from which HMOs are built are selected from the group consisting of D-glucose, D-galactose, N-acetyl-D-glucosamine, L-fucose and N-acetylneuraminic acid.

Additionally and/or alternatively, the oligosaccharide of interest is a HMO selected from the group consisting of 2′-fucosyllactose (2′-FL), 3-fucosyllactose (3-FL), 2′,3-difucosyllactose (DFL), lacto-N-triose II, lacto-N-tetraose (LNT), lacto-N-neotetraose (LNnT), lacto-N-fucopentaose I (LNFP-I), lacto-N-neofucopentaose I (LNnFP-I), lacto-N-fucopentaose II (LNFP-II), lacto-N-fucopentaose III (LNFP-III), lacto-N-fucopentaose V (LNFP-V), lacto-N-neofucopentaose V (LNnFP-V), lacto-N-hexaose (LNH), lacto-N-neohexaose (LNnH), para-lacto-N-hexaose (paraLNH), para-lacto-N-neohexaose (paraLNnH), difucosyl-lacto-N-neohexaose (DF-LNnH), lacto-N-difucosylhexaose I, lacto-N-difucosylhexaose II, para-lacto-N-fucosylhexaose (paraLNH), fucosyl-lacto-N-sialylpentaose a (F-LST-a), fucosyl-lacto-N-sialylpentaose b (F-LST-b), fucosyl-lacto-N-sialylpentaose c (F-LST-c), fucosyl-lacto-N-sialylpentaose c, disialyl-lacto-N-fucopentaose, 3-fucosyl-3′-sialyllactose (3F-3′-SL), 3-fucosyl-6′-sialyllactose (3F-6′-SL), lacto-N-neodifucohexaose I, 3′-sialyllactose (3-SL), 6′-sialyllactose (6-SL), sialyllacto-N-tetraose a (LST-a), sialyllacto-N-tetraose b (LST-b), sialyllacto-N-tetraose c (LST-c), disialyllacto-N-tetraose (DS-LNT), Disialyl-lacto-N-fucopentaose (DS-LNFP V), lacto-N-neodifucohxaose I (LNnDFH I), 3′-galactosyllactose (3′-GL), 6′-galactosyllactose (6′-GL).

The genetically engineered microbial cell is a microbial cell that has been genetically engineered to possess the metabolic pathway(s) for an intracellular biosynthesis of the oligosaccharide of interest. Hence, the genetically engineered microbial cell is capable of intracellularly synthesizing the oligosaccharide of interest.

The terms “capable of” and “able to” with respect to the biosynthesis of the oligosaccharide of interest by the genetically engineered microbial cell as used herein means that the genetically engineered mcirobial cell synthesizes the oligosaccharide of interest intracellularly when the microbial cell is cultured in a medium and at conditions that are permissive (e.g. with respect to temperature, pH, osmolarity, nutritional composition) for the intracellular biosynthesis of the oligosaccharide of interest.

The genetically engineered microbial cell is able to synthesize the oligosaccharide of interest intracellularly when being cultivated in the presence of exogenous glycerol as sole carbon/energy source. Therefore, the genetically engineered microbial cell possesses a glycerol permease for internalization of exogenous glycerol. The glycerol permease, also designated as a glycerol uptake facilitator, internalizes exogenously supplied glycerol.

In some embodiments, the glycerol permease is an endogenous glycerol permease of the microbial cell, i.e. a glycerol permease that is naturally occurring in native cells of the same species the genetically engineered microbial cell belongs to. In additional and/or alternative embodiments, the glycerol permease is a heterologous glycerol permease, i.e. a glycerol permease that is not naturally occurring in native cells of the microbial species the genetically engineered microbial cell belongs to.

Additionally and/or alternatively, the genetically engineered microbial cell has been genetically engineered to possess a glycerol permease or to possess an increased glycerol permease activity as compared to the progenitor cell that has not yet been genetically engineered to possess an increased glycerol permease activity. Upon the genetical engineering of the microbial cell to possess a glycerol permease or an increased glycerol permease activity, a glycerol permease is expressed from an exogenous glycerol permease-encoding gene that has been inserted into the microbial cell, and/or by genetic modification of the microbial cell's endogenous glycerol permease-coding gene(s). In some of these embodiments, a progenitor cell of the genetically engineered microbial cell has been genetically engineered to contain the exogenous glycerol permease-encoding gene, and to express the glycerol permease. In some embodiments, the exogenous glycerol permease-encoding gene is integrated into the or at least into one of the microbial cell's chromosome(s). Additionally and/or alternatively, the exogenous glycerol permease-encoding gene is present on an episomal nucleic acid molecule within the microbial cell. In some embodiments, the glycerol permease-encoding gene is a recombinant gene.

An example of a suitable glycerol permease is the glycerol uptake faciliator protein GlpF of E. coli (strain K-12) as encoded by the E. coli glpF gene. The deduced amino acid sequence of E. coli K-12 GlpF is disclosed in the UniProt Knowledge base (www.uniprot.org), release 2022_01 as of Feb. 23, 2022: entry number POAERO. Hence, in some embodiments, the genetically engineered microbial cell possess an E. coli GlpF or a functional variant thereof. Additionally and/or alternatively, the microbial cell has been genetically engineered to possess an E. coli K-12 glycerol permease GlpF or a functional variant thereof. Thus, in some embodiments, the microbial cell is a cell that has been genetically engineered to possess a recombinant gene comprising and expressing a nucleotide sequence that encodes E. coli GlpF or a functional variant thereof.

The term “variant(s)” as used herein, refers to a polynucleotide or polypeptide that differs in its nucleotide sequence or amino acid sequence from the sequence of a reference polynucleotide or polypeptide respectively, but retains the essential properties (e.g. catalytic) of the reference polynucleotide or polypeptide. A typical variant of a polynucleotide differs in nucleotide sequence from another polynucleotide being the reference polynucleotide. Differences in the nucleotide sequence of the variant may or may not alter the amino acid sequence of a polypeptide encoded by the reference polynucleotide. Nucleotide deviations may result in amino acid substitutions, additions, deletions, fusions and/or truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be naturally occurring such as being an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

Within the scope of the present invention, also nucleic acid/polynucleotide and polypeptide polymorphic variants, alleles, mutants, and interspecies homologs are comprised by those terms, that possess a nucleotide sequence or an amino acid sequence that has greater than about 60% sequence identity, 65%, 70%, 75%, 80%, 85%, 90%, 91%, 92%, 93%, 94%, 95%, 96%, 97%, 98% or 99% or greater sequence identity to the reference sequence. The sequence identity extends over a region of at least about 25, 50, 100, 200, 500, 1000, or more nucleotides or amino acids, as compared to the reference sequence.

A “variant” of any gene/polypeptide is meant to designate sequence variants of the gene/protein which retain the same, a lower or a higher activity of the gene, the polypeptide and/or the polypeptide that is encoded by the gene, or a different activity in regard to the substrate specificity of the reference polypeptide, or a different activity in regard to the reaction specificity of the reference polypeptide.

The term “operon” as used herein refers to a nucleotide sequence comprising two or more protein coding sequences that are transcribed from the same regulatory element for mediating and/or controlling expression in the microbial cell.

The term “functional gene” as used herein refers to a nucleic acid molecule comprising a nucleotide sequence which encodes a protein or polypeptide, and which also contains regulatory sequences operably linked to said protein-coding nucleotide sequence (open reading frame) such that the nucleotide sequence which encodes the protein or polypeptide can be expressed in/by the microbial cell bearing the functional gene. Thus, when cultured at conditions that are permissive for the expression of the functional gene, said gene is expressed, and the microbial cell expressing the functional gene typically comprises the protein or polypeptide that is encoded by the protein coding region of the functional gene. As used herein, the terms “nucleic acid” and “polynucleotide” refer to a deoxyribonucleotide or ribonucleotide polymer in either single- or double-stranded form, and unless otherwise limited, encompasses known analogues of natural nucleotides that hybridize to nucleic acids in a manner like naturally occurring nucleotides.

The term “deregulated” as used herein with respect to the expression of endogenous genes refers to an altered expression of the protein coding region of the endogenous gene(s) as compared to the expression of the gene(s) in the native progenitor of the genetically engineered microbial cell which progenitor cell has not been genetically engineered to alter the expression of the gene(s). The term “deregulated” comprises an increased expression of a gene as well as a decreased or impaired expression of the gene. Expression of a gene can be deregulated by different means known to the skilled artisan. Examples of deregulating the expression of a gene include alterations of the native nucleotide sequence of the gene's promoter, alteration of the native nucleotide sequence of the gene's ribosomal binding site.

The term “operably linked” as used herein shall mean a functional linkage between a nucleotide sequence which controls expression (such as a promoter, signal sequence, or array of transcription factor binding sites) and a second nucleotide sequence, wherein the nucleotide sequence controlling expression effects transcription and/or translation of the second nucleotide sequence. Accordingly, the term “promoter” designates a nucleotide sequence which usually “precedes” a protein coding nucleotide sequence of a DNA polynucleotide and provides a site for initiation of the transcription into mRNA. “Regulator” DNA sequences, also usually “upstream” of (i.e., preceding) the protein-coding nucleotide sequence of a gene in a given DNA polymer, bind proteins that determine the frequency (or rate) of transcriptional initiation. Collectively referred to as “promoter/regulator” or “control” DNA sequence, these sequences which precede a selected gene (or series of genes) in a functional DNA polymer cooperate to determine whether the transcription (and eventual expression) of a gene will occur. Nucleotide sequences which “follow” a protein coding nucleotide sequence in a DNA polymer and provide a signal for termination of the transcription into mRNA are referred to as transcription “terminator” sequences.

The term “recombinant”, as used herein indicates that a polynucleotide, such as a gene or operon, has been created by means of genetic engineering. Hence, a recombinant gene, operon or polynucleotide is not naturally occurring in microbial cells of the same species as the microbial cell for the production of the oligosaccharide of interest. A recombinant gene or operon comprise nucleotide sequences originating from at least two different ancestries such as e.g. from different loci or from different species. An example is the protein-coding region of a glycerol permease-encoding gene operably linked to the promoter of a different gene. The recombinant polynucleotide may contain a heterologous nucleotide sequence, or expresses a polypeptide encoded by a heterologous nucleotide sequence (i.e., a nucleotide sequence that is foreign to said microbial cell). Recombinant microbial cells can contain genes that are not found in its native (non-recombinant) progenitor. Recombinant cells can also contain variants of genes that are found in the recombinant cell's native progenitor, wherein the genes were modified and re-introduced into the cell by technical means. The term “recombinant” also encompasses microbial cells that contain a nucleotide sequence that is endogenous to the bacterial cell and has been modified without removing the nucleic acid molecule bearing said nucleotide sequence from the bacterial cell. Such modifications include those obtained by gene replacement, site-specific mutation, and related techniques. Accordingly, a “recombinant polypeptide” is one which has been produced by a recombinant cell.

A “heterologous nucleotide sequence” or a “heterologous nucleic acid”, as used herein, is one that originates from a source foreign to the particular host cell (e.g. from a different species), or, if from the same source, is modified from its original form. Thus, a heterologous nucleotide sequence may be a nucleotide sequence wherein a heterologous protein-coding nucleotide sequence being operably linked to a promoter, wherein the protein-coding nucleotide sequence and the nucleotide sequence of the promoter were obtained from different source organisms, or, if from the same source organism, either the protein-coding nucleotide sequence or the promoter has been modified from its original form. The heterologous nucleotide sequence may be stably introduced into the genome of the bacterial cell, e.g. by transposition, transfection, transformation, conjugation or transduction. The techniques that may be applied will depend on the microbial cell and the specificities of the nucleic acid molecule to be introduced into the microbial cell. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). Accordingly, a “genetically engineered microbial cell” is understood as a microbial cell which has been transformed or transfected, or is capable of transformation or transfection by an exogenous polynucleotide sequence. Thus, the nucleotide sequences as used in the invention, may, e.g., be comprised in a vector which is to be stably transformed/transfected or otherwise introduced into host microorganism cells. A great variety of vectors can be used to have polypeptides synthesized in the microbial cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and to synthesize a polypeptide in a bacterial host cell may be used for expression in this regard. The appropriate nucleotide sequence may be inserted into the expression system by any of a variety of well-known and routine techniques, such as, for example, those set forth in Sambrook et al., supra.

Preferably, polynucleotides containing the recombinant nucleotide sequence are stably introduced into the genome of the microbial cell. Genomic integration can be achieved by recombination or transposition.

The genetically engineered microbial cell comprises a glycerol kinase. A glycerol kinase is an enzyme that catalyzes the phosphorylation of glycerol using adenosine triphosphate (ATP) to yield sn-glycerol 3-phosphate. More specifically, the microbial cell possesses a glycerol kinase which is a functional variant of the E. coli K12 glycerol kinase GlpK.

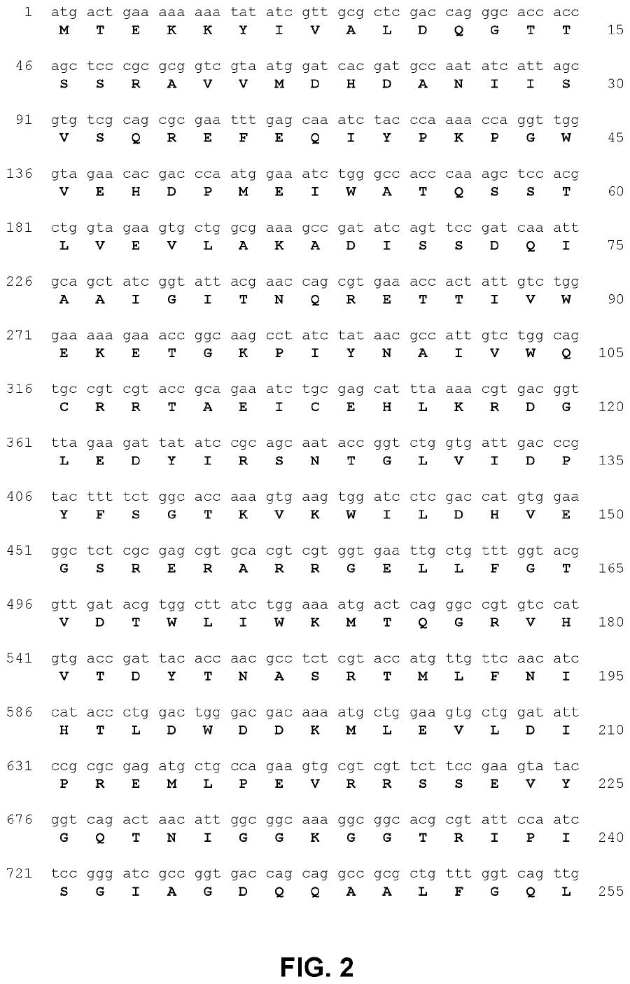

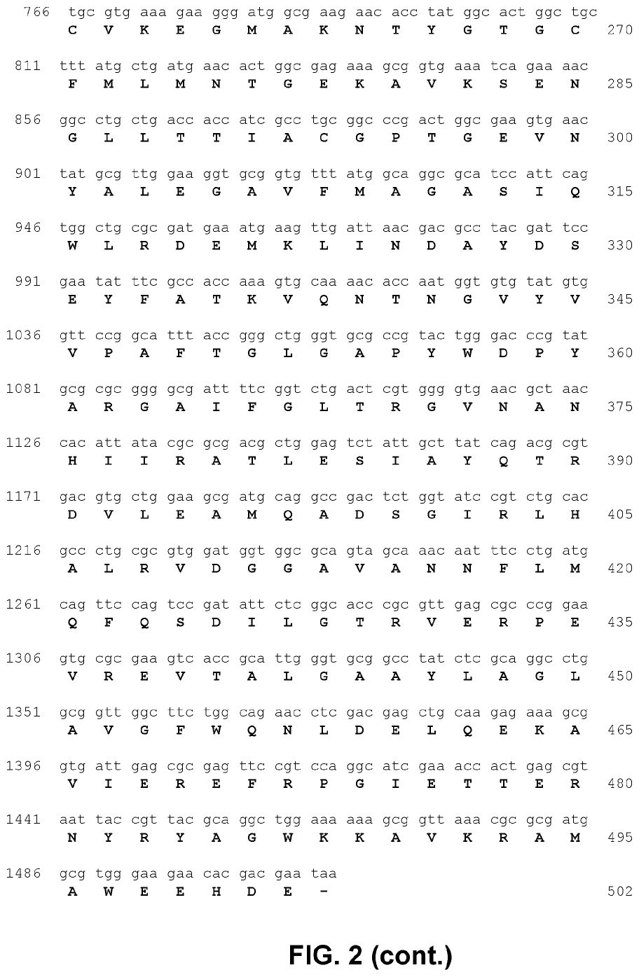

The glycerol kinase of E. coli (strain K12) is encoded by the protein-coding nucleotide sequence of the E. coli (K12) glpK gene. The nucleotide sequence of the protein coding region of the E. coli (K12) glpK gene is set forth in SEQ ID NO: 2 of the attached sequence listing. The deduced amino acid sequence of E. coli K-12 GlpK is disclosed in the UniProt Knowledge base (www.uniprot.org), release 2022_01 as of Feb. 23, 2022: entry number POA6F3. In addition or alternative, the amino acid sequence of the native E. coli K12 glycerol kinase GlpK is also set forth in SEQ ID NO: 1 of the sequence listing. FIG. 2 illustrates the nucleotide sequence of the protein-coding region of E. coli (K12) glpK and the E. coli (K12) GlpK amino acid sequence deduced from the nucleotide sequence.

In certain embodiments, the functional variant of the E. coli (K12) glycerol kinase GlpK comprises an amino acid residue within its amino acid sequence at the position which corresponds to amino acid position 55 in the amino acid sequence of E. coli (K12) GlpK as set forth in SEQ ID NO: 1, wherein said amino acid residue comprises a non-ionized but polar acting side chain, and/or the variant comprises an amino acid residue at its position which corresponds to amino acid position 231 in the amino acid sequence of E. coli (K12) GlpK as set forth in SEQ ID NO: 1, wherein said amino acid residue comprises an anionic side chain.

Hence, the microbial cell comprises a glycerol kinase, wherein said glycerol kinase is a functional variant of the E. coli glycerol kinase GlpK, wherein said functional variant possesses an amino acid residue at its position which corresponds to amino acid position 55 in the amino acid sequence of E. coli GlpK as set forth in SEQ ID NO: 1, wherein said amino acid residue comprises a non-ionized (also called uncharged) but polar acting side chain.

Amino acids which possess a non-ionized but polar acting side chain belong to the group of amino acids having a side chain which possesses a functional group that comprises at least one atom with electron pairs available for hydrogen bonding to water.

In some embodiments, the amino acid residue comprising the non-ionized but polar acting side chain is a hydrophilic amino acid.

Additionally and/or alternatively, the amino acid comprising a non-ionized but polar acting side chain is an amidic amino acid. The amidic amino acid is preferably selected from the group consisting of L-asparagine and L-glutamine.

Additionally and/or alternatively, the amino acid comprising a non-ionized but polar acting side chain is a sulphur-containing amino acid. The sulphur-containing amino acid is preferably selected from the group consisting of L-serine, L-threonine, L-cysteine and L-methionine.

Additionally and/or alternatively, the amino acid residue that comprises a non-ionized but polar acting side chain is an amino acid that is selected from the group of amino acids consisting of L-serine, L-threonine, L-cysteine, L-methionine, L-asparagine and L-glutamine.

Additionally and/or alternatively, the microbial cell comprises a glycerol kinase, wherein said glycerol kinase is a functional variant of the E. coli (K12) glycerol kinase GlpK, wherein said functional variant possesses an amino acid residue at the position which corresponds to amino acid position 231 in the amino acid sequence of E. coli GlpK as set forth in SEQ ID NO: 1, wherein said amino acid residue comprises an anionic side chain. The amino acid residue that comprises an anionic side chain is preferably selected from the group consisting of amino acids L-aspartate and L-glutamate.

For possessing any one of said functional variants of the E. coli (K12) glycerol kinase GlpK, a progenitor cell of the genetically engineered microbial cell is genetically engineered to comprise a nucleic acid molecule that encodes and expresses a nucleotide sequence which encodes at least one of the functional variants. Thus, the genetically engineered microbial cell comprises an exogenous functional gene encoding and expressing the functional variant of the E. coli (K12) glycerol kinase GlpK.

In some embodiments, the exogenous gene encoding the functional variant of E. coli (K12) GlpK is integrated into the microbial cell's chromosome or integrated into at least one of the microbial cell's chromosomes. Additionally and/or alternatively, the recombinant functional gene encoding said functional variant of the E. coli (K12) glycerol kinase GlpK is comprised by an episomal nucleic acid molecule that is present in the genetically engineered microbial cell.

In some embodiments, the microbial cell has been genetically engineered in that the nucleotide sequence of the microbial cell's endogenous glycerol kinase gene has been altered such that the resulting glycerol kinase-encoding gene encodes the functional variant of E. coli (K12) GlpK.

The genetically engineered microbial cell possesses a glycerol kinase which is one of the functional variants of the E. coli (K12) glycerol kinase GlpK described herein. The microbial cell does not possess a native or endogenous glycerol kinase which is not one of the functional variants of the E. coli (K12) glycerol kinase GlpK and which possesses a higher enzymatic activity than any one of the functional variants of the E. coli (K12) glycerol kinase GlpK described herein. Thus, in some embodiments, the genetically engineered microbial cell comprises a deletion or functional inactivation of its endogenous gene(s) encoding a glycerol kinase.

The functional inactivation may be a deletion and/or inactivation of one or more expression control sequences of the microbial cell's native glycerol kinase gene(s) such that the expression of the microbial cell's native glycerol kinase gene(s) is abolished. Additionally or alternatively, said functional inactivation may be a deletion or alteration of the protein coding region of the microbial cell's native glycerol kinase gene(s) such as, e.g. by introducing frame shift mutations, mutations impairing translation initiation, mutations introducing stop codons or changes is the amino acid sequence of the polypeptide encoded by the altered nucleotide sequence such that the resulting polypeptide does not possess glycerol kinase activity.

In some embodiments, the genetically engineered microbial cell comprises a functional exogenous gene encoding a functional variant of the E. coli (K12) glycerol kinase GlpK as disclose herein in addition to the deletion or functional inactivation of the microbial cell's endogenous glycerol kinase gene(s).

In some embodiments, the genetically engineered microbial cell has been genetically engineered such that the protein coding region of its native glycerol kinase gene has been altered to encode the functional variant of the E. coli (K12) GlpK. Alteration of the protein coding region of a microbial cell's native or endogenous glycerol kinase gene is an option when the genetically engineered microbial cell is an E. coli cell.

In some embodiments, the microbial cell is a genetically engineered microbial cell that has been genetically engineered in that the exogenous gene encoding the functional variant of the E. coli (K12) glycerol kinase replaced the native/endogenous glycerol kinase gene(s) of the microbial cell. Replacement of the protein coding region of a microbial cell's native or endogenous glycerol kinase gene is an alternative option to the alteration of the protein-coding region of the microbial cell's native or endogenous glycerol kinase-encoding gene when the genetically engineered microbial cell is an E. coli cell.

The genetically engineered microbial cell further comprises a metabolic pathway for the intracellular biosynthesis of the oligosaccharide of interest. Hence, the genetically engineered microbial cell comprises a metabolic pathway for intracellular biosynthesis of a nucleotide-activated monosaccharide as a donor substrate of its monosaccharide moiety for being transferred to an acceptor molecule, all enzymes and transporters that are necessary to provide an acceptor molecule for acquiring the monosaccharide moiety, and a glycosyltransferase for transfer of the monosaccharide moiety from the nucleotide-activated monosaccharide to the acceptor molecule.

The genetically engineered microbial cell comprises a metabolic pathway for intracellular synthesis of a nucleotide-activated monosaccharide. Said nucleotide-activated monosaccharide serves as a donor substrate of its monosaccharide moiety, wherein a glycosyltransferase transfers the monosaccharide moiety from the donor substrate to an acceptor molecule.

In some embodiments, the nucleotide-activated monosaccharide is selected from the group consisting of guanosine-5′-diphospho-β-L-fucose (GDP-Fuc), cytidine-5′-monophospho-N-acetylneuraminic acid (CMP-NeuNAc), uridine-5′-diphospho-α-D-galactose (UDP-Gal), uridine-5′-disphospho-N-acetylglucosamine (UDP-GIcNAc), and uridine-5′-diphospho-α-D-glucose (UDP-Glc).

The nucleotide-activated monosaccharide may be synthesized by the genetically engineered microbial cell in a de novo pathway. Additionally and/or alternatively, the microbial cell may use a salvage pathway for providing the nucleotide-activated monosaccharide.

In embodiments wherein the nucleotide-activated monosaccharide is synthesized by a de novo pathway, the microbial cell possesses the enzymes that are necessary for the de novo biosynthesis pathway of the nucleotide-activated monosaccharide. In some embodiments, the microbial cell has been genetically engineered to possess at least one exogenous gene encoding an enzyme that is necessary for the de novo biosynthesis pathway for the nucleotide-activated monosaccharide.

The at least one gene encoding an enzyme that is necessary for the de novo biosynthesis pathway of the nucleotide-activated monosaccharide may be endogenous to the microbial cell or they can be introduced from exogenous sources into the microbial cell to be expressed. Expression, i.e. functional transcription and translation, of proteins encoded by endogenous genes can be modified by genetically engineering the microbial cell. For example, alteration of the expression of an endogenous gene may be achieved by modifying the gene's transcriptional promotor, by modifying the gene's ribosome binding site and/or by modifying the codon usage of the gene's protein-coding nucleotide sequence. In addition, the protein-coding nucleotide sequence of the gene may be modified such that the activity and/or the specificity of the enzyme being encoded by said gene becomes favorable for the desired biosynthesis pathway. A person skilled in the art would know which gene(s) need to be expressed in the genetically modified organism for de novo synthesis of nucleotide-activated sugar donor molecules.

In embodiments wherein a salvage pathway is used for the biosynthesis of the nucleotide-activated monosaccharide as donor substrate, the microbial cell comprises an enzyme which catalyzes the linkage of a nucleotide and a monosaccharide. An example of such an enzyme is the bifunctional fucokinase/L-fucose-1-phosphate-guanylyltransferase FKP from Bacteroides fragilis which catalyzes a kinase reaction and a pyrophosphatase reaction to form GDP-L-fucose. A further example of an enzyme which catalyzes the linkage of a nucleotide and a monosaccharide is the N-acetylneuraminate cytidyltransferase NeuA from Neisseria meningitidis which forms CMP-neuraminic acid.

In some embodiment, the microbial cell possessing a salvage pathway for providing a nucleotide-activated monosaccharide as donor substrate contains an exogenous gene encoding and expressing the enzyme which catalyzes the linkage of the nucleotide and the monosaccharide.

A monosaccharide that is used as substrate for the formation of the nucleotide-activated monosaccharide in the salvage pathway may be internalized by the microbial cell utilizing a monosaccharide import protein. Examples of such monosaccharide import proteins are a fucose permease, e.g. FucP, and a sialic acid importer, e.g. NanT from E. coli. The monosaccharide import proteins may be encoded by and expressed from an endogenous gene or may be encoded by and expressed from an exogenous gene.

For the biosynthesis of the oligosaccharide of interest, the acceptor molecule is a saccharide, i.e. an acceptor saccharide. The acceptor molecule may be selected from the group consisting of monosaccharides, disaccharides and oligosaccharides. In some embodiments, the acceptor saccharide is a disaccharide that is converted into a trisaccharide. In other embodiments, the acceptor saccharide is a trisaccharide that is converted into a tetrasaccharide. In other embodiments, the acceptor saccharide is a tetrasaccharide that is converted into a pentasaccharide. In some embodiments, the acceptor saccharide is a pentasaccharide that is converted into a hexasaccharide.

In some embodiments, the acceptor saccharide or a precursor of the acceptor saccharide is internalized by the microbial cell, preferably by utilizing a specific transporter that is present in the microbial cell's cell membrane. In the process of producing the oligosaccharide of interest using the genetically engineered microbial cell, the genetically engineered microbial cell is cultured in the presence of the acceptor saccharide or in the presence of a precursor of the acceptor saccharide. The acceptor saccharide or its precursor present in the culture medium is internalized by the genetically engineered microbial cell, and utilized for the biosynthesis of the oligosaccharide of interest.

Additionally and/or alternatively the acceptor saccharide is synthesized by the genetically engineered microbial cell intracellularly.

In some embodiments, the acceptor saccharide is synthesized intracellularly by the microbial cell from a monosaccharide that has been internalized by the microbial cell and is elongated by a monosaccharide moiety to obtain a disaccharide, wherein said disaccharide may be further elongated by the addition of further monosaccharide moieties such that an oligosaccharide is synthesized intracellularly which consists of three, four, five, six or more monosaccharide moieties, which then constitutes the acceptor saccharide for the biosynthesis of the oligosaccharide of interest. For example, the microbial cell internalizes glucose which is converted to lactose by the addition of a galactose moiety. Said lactose may then be converted to an oligosaccharide such as, e.g., 2′-FL, 3-FL, LNT-II, LNT, LNnT, 3′-SL, 6′-SL. Each of said oligosaccharides may be an oligosaccharide of interest or may constitute an acceptor saccharide.

In some embodiments, the microbial cell has been genetically engineered to possess the enzyme which catalyze the conversion of the acceptor saccharide, i.e. a disaccharide or an oligosaccharide, to the oligosaccharide of interest by transferring the monosaccharide moiety from the donor substrate to the acceptor saccharide. Hence, the microbial cell contains and expresses at least one exogenous gene, a homologous or a heterologous gene, which encodes an enzyme that catalyzes the conversion of the acceptor saccharide to the oligosaccharide of interest.

Genes or equivalent functional nucleotide sequences encoding the enzymatic ability of the microbial cell that catalyzes the conversion of the acceptor saccharide to the oligosaccharide of interest, i.e. adds a monosaccharide moiety from the donor substrate to the acceptor saccharide, may be a homologous nucleotide sequence or a heterologous nucleotide sequence. The term “homologous” as used herein with respect to nucleotide sequences refers to nucleotide sequences that are native to the species the microbial cell for producing the oligosaccharide of interest belongs to. The term “heterologous” as used herein with respect to nucleotide sequences refers to nucleotide sequences that are artificially generated or derived from a different species than the microbial cell for producing the oligosaccharide of interest belongs to. Heterologous nucleotide sequences that are derived from a different species than the one of the microbial cell for producing the oligosaccharide of interest may originate from a plant, an animal including humans, a bacterium, an archaea, a fungus or a virus.

The enzyme catalyzing the transfer of a monosaccharide moiety to the acceptor molecule is a glycosyltransferase or a transglycosidase. While a transglycosidase catalyzes the transfer of a monosaccharide moiety from one saccharide to another saccharide, the glycosyltransferases catalyze the transfer of a monosaccharide moiety from a nucleotide-activated sugar as donor substrate to the acceptor saccharide. The glycosyltransferase may be selected from the group consisting of galactosyltransferases, glucosyltransferases, fucosyltransferases, sialyltransferases, N-acetylglucosaminyltransferases, N-acetylgalactosaminyltransferases, glucuronosyltransferases, mannosyltransferases, and xylosyltransferases. Additionally and/or alternatively, the glycosyltransferase is selected from the group consisting of 3-1,3-galactosyltransferases, 3-1,4-galactosyltransferases, β-1,6-galactosyltransferases, α-1,3-glucosyltransferases, α-1,4-glucosyltransferases, α-1,2-fucosyltransferases and α-1,3-fucosyltransferases, α 1,4-fucosyltransferases, α-2,3-sialyltransferases, α-2,6-sialyltansferases, α-2,8-sialyltansferases, β-1,3-N-acetylglucosaminyltransferases, β-1,4-N-acetylglucosaminyltransferases, α-1,3-N-acetylgalactosaminyltransferases, β-1,3-N-acetylgalactosaminyltransferases, β-1,4-N-acetylgalactosaminyltransferases, α-1,2-mannosyltransferases, β-1,4-xylosyltransferases.

It is understood that a specific glycosyltransferase catalyzes the transfer of the respective monosaccharide moiety from the nucleotide-activated monosaccharide to the acceptor molecule.

That said, some genetically engineered microbial cells possess a metabolic pathway for providing GDP-Fuc and further possess a fucosyltransferase, for the biosynthesis of a fucosylated oligosaccharide of interest such as e.g. 2′-fucosyllactose (2′-FL), 3-fucosyllactose (3-FL), 2′,3-difucosyllactose (DFL), lacto-N-fucopentaose I (LNFP-I), lacto-N-neofucopentaose I (LNnFP-I), lacto-N-fucopentaose II (LNFP-II), lacto-N-fucopentaose III (LNFP-III), lacto-N-fucopentaose V (LNFP-V), lacto-N-neofucopentaose V (LNnFP-V), difucosyl-lacto-N-neohexaose (DF-LNnH), lacto-N-difucosylhexaose I, lacto-N-difucosylhexaose II, para-lacto-N-fucosylhexaose (paraLNH), fucosyl-lacto-N-sialylpentaose a (F-LST-a), fucosyl-lacto-N-sialylpentaose b (F-LST-b), fucosyl-lacto-N-sialylpentaose c (F-LST-c), fucosyl-lacto-N-sialylpentaose c, disialyl-lacto-N-fucopentaose, 3-fucosyl-3′-sialyllactose (3F-3′-SL), 3-fucosyl-6′-sialyllactose (3F-6′-SL), lacto-N-neodifucohexaose I, disialyl-lacto-N-fucopentaose (DS-LNFP V), or lacto-N-neodifucohxaose I (LNnDFH I).

Some genetically engineered microbial cells possess a metabolic pathway for providing CMP-NeuNAc and further possess a sialyltransferase, for the biosynthesis of a sialylated oligosaccharide of interest such as e.g. 3′-sialyllactose (3′-SL), 6′-sialyllactose (6′-SL), sialyllacto-N-tetraose a (LST-a), sialyllacto-N-tetraose b (LST-b), sialyllacto-N-tetraose c (LST-c), disialyllacto-N-tetraose (DS-LNT), fucosyl-lacto-N-sialylpentaose a (F-LST-a), fucosyl-lacto-N-sialylpentaose b (F-LST-b), fucosyl-lacto-N-sialylpentaose c (F-LST-c), fucosyl-lacto-N-sialylpentaose c, disialyl-lacto-N-fucopentaose, 3-fucosyl-3′-sialyllactose (3F-3′-SL), 3-fucosyl-6′-sialyllactose (3F-6′-SL), lacto-N-neodifucohexaose I, Disialyl-lacto-N-fucopentaose (DS-LNFP V), or lacto-N-neodifucohxaose I (LNnDFH I).

Some genetically engineered microbial cells possess a metabolic pathway for providing UDP-GIcNAc and further possess a N-acetylglucosaminyltransferase, for the biosynthesis of an N-acetylglucosamine-containing oligosaccharide such as e.g. lacto-N-triose II, lacto-N-tetraose (LNT), lacto-N-neotetraose (LNnT), lacto-N fucopentaose I (LNFP-I), lacto-N-neofucopentaose I (LNnFP-I), lacto-N-fucopentaose II (LNFP-II), lacto-N-fucopentaose III (LNFP-III), lacto-N-fucopentaose V (LNFP-V), lacto-N-neofucopentaose V (LNnFP-V), lacto-N-hexaose (LNH), lacto-N-neohexaose (LNnH), para-lacto-N-hexaose (paraLNH), para-lacto-N-neohexaose (paraLNnH), difucosyl-lacto-N-neohexaose (DF-LNnH), lacto-N-difucosylhexaose I, lacto-N-difucosylhexaose II, para-lacto-N-fucosylhexaose (paraLNH), fucosyl-lacto-N-sialylpentaose a (F-LST-a), fucosyl-lacto-N-sialylpentaose b (F-LST-b), fucosyl-lacto-N-sialylpentaose c (F-LST-c), fucosyl-lacto-N-sialylpentaose c, disialyl-lacto-N-fucopentaose, 3-fucosyl-3′-sialyllactose (3F-3′-SL), 3-fucosyl-6′-sialyllactose (3F-6′-SL), lacto-N-neodifucohexaose I, sialyllacto-N-tetraose a (LST-a), sialyllacto-N-tetraose b (LST-b), sialyllacto-N-tetraose c (LST-c), disialyllacto-N-tetraose (DS-LNT), Disialyl-lacto-N-fucopentaose (DS-LNFP V), or lacto-N-neodifucohxaose I (LNnDFH I).

Some genetically engineered microbial cells possess a metabolic pathway for providing UDP-Gal and further possess a galactosyltransferase, for the biosynthesis of a galactose moiety containing oligosaccharide such as e.g. of 2′-fucosyllactose (2′-FL), 3-fucosyllactose (3-FL), 2′,3-difucosyllactose (DFL), lacto-N-triose II, lacto-N-tetraose (LNT), lacto-N-neotetraose (LNnT), lacto-N-fucopentaose I (LNFP-I), lacto-N-neofucopentaose I (LNnFP-I), lacto-N-fucopentaose II (LNFP-II), lacto-N-fucopentaose III (LNFP-III), lacto-N-fucopentaose V (LNFP-V), lacto-N-neofucopentaose V (LNnFP-V), lacto-N-hexaose (LNH), lacto-N-neohexaose (LNnH), para-lacto-N-hexaose (paraLNH), para-lacto-N-neohexaose (paraLNnH), difucosyl-lacto-N-neohexaose (DF-LNnH), lacto-N-difucosylhexaose I, lacto-N-difucosylhexaose II, para-lacto-N-fucosylhexaose (paraLNH), fucosyl-lacto-N-sialylpentaose a (F-LST-a), fucosyl-lacto-N-sialylpentaose b (F-LST-b), fucosyl-lacto-N-sialylpentaose c (F-LST-c), fucosyl-lacto-N-sialylpentaose c, disialyl-lacto-N-fucopentaose, 3-fucosyl-3′-sialyllactose (3F-3′-SL), 3-fucosyl-6′-sialyllactose (3F-6′-SL), lacto-N-neodifucohexaose I, 3′-sialyllactose (3′-SL), 6′-sialyllactose (6′-SL), sialyllacto-N-tetraose a (LST-a), sialyllacto-N-tetraose b (LST-b), sialyllacto-N-tetraose c (LST-c), disialyl-lacto-N-tetraose (DS-LNT), Disialyl-lacto-N-fucopentaose (DS-LNFP V), lacto-N-neodifucohxaose I (LNnDFH I), 3′-galactosyllactose (3′-GL), 6′-galactosyllactose (6′-GL).

In some embodiments, the genetically engineered microbial cell possesses a saccharide transporter in its cell membrane. The saccharide transporter translocates the oligosaccharide of interest from the cytoplasm of the microbial cell across its cell membrane into the culture medium or—in case of gram-negative bacteria—into the periplasmic space.

A saccharide transporter mediating the translocation of a carbohydrate, e.g. the oligosaccharide of interest, across the cell membrane may consist of a single polypeptide or may consist of multiple polypeptides (each one considered being a subunit) which forming a homomeric or heteromeric complex that translocates the oligosaccharide of interest across the cell membrane.

In some embodiments, the saccharide transporter is a member selected from the group consisting of the major facilitator superfamily (MFS), the sugar: cation symporter family, the nucleoside-specific transporter family, the ATP-binding cassette superfamily, and the phosphotransferase system family.

In certain embodiments, the saccharide transporter is encoded by and expressed from an exogenous gene. Hence, in such embodiments, the microbial cell comprises an exogenous gene which encodes the saccharide transporter or at least one subunit of a saccharide transporter complex.

The exogenous gene(s) encoding the transporter or the transporter complex facilitating the transport of the oligosaccharide of interest across the cell membrane can either be endogenous or exogenous to the genetically engineered microbial cell.

In some embodiments the saccharide transporter gene is endogenous to the genetically engineered bacterial cell. Functional elements effecting synthesis of the protein that is encoded by the open reading frame of the saccharide transporter gene(s) may have been modified such that the level of transcription of the open reading frame or translation of the mRNA leading to synthesis of the transporter protein is different from that naturally found in the predecessor cell. Modification of the functional elements can either increase the transcription and/or translation level or reduce the transcription and/or translation level compared to the unmodified state. Modifications can be single nucleotide modifications or exchange of complete functional elements. In either case the non-natural level of transcription and/or translation is optimized for the transport of the intracellular oligosaccharide of interest across the inner membrane.

In another embodiment the gene or genes encoding the saccharide transporter or transporter complex is/are exogenous to the genetically engineered microbial cell. In such embodiments, the protein coding region is operably linked to naturally occurring or artificial functional elements such as promoters or ribosome binding sites in a way that the transcription level and the translation level of the transporter is optimized for the transport of the oligosaccharide of interest across the inner membrane.

Transcription of the gene(s) encoding the saccharide transporter or the subunits of the saccharide transporter can either be constitutive or regulated. A person skilled in the art would know how to choose and use a promoter and/or a promoter in combination with a further transcriptional regulator to achieve optimal expression of the gene(s) encoding the saccharide transporter.

In embodiments wherein the gene(s) encoding the saccharide transporter is exogenous to the microbial cell for the production of the oligosaccharide of interest, the nucleotide sequence of the gene(s) may be modified as compared to the naturally occurring nucleotide sequence, but may encode a polypeptide possessing an unaltered amino acid sequence.

The saccharide transporter for translocating the oligosaccharide of interest across the cell membrane can have its native amino acid sequence, i.e. the amino acid sequence that is naturally found. Alternatively, the amino acid sequence of the saccharide transporter can be modified as compared to its native amino acid sequence such that the resulting variant has enhanced activity with respect to the translocation of the oligosaccharide of interest and/or may have other beneficial characteristics, e.g. enhanced protein stability or more stable integration into the inner membrane.

In particular embodiments, the nucleotide sequence encoding the saccharide transport protein(s) is artificial and accordingly, the amino acid sequence of the resulting protein(s) acting as transporter to export the oligosaccharide of interest from the cytoplasm into the periplasm is as such not found in nature.

In some embodiments, the genetically engineered microbial cell further comprises a deletion or functional inactivation of its endogenous methylglyoxal synthase gene. The methylglyoxal synthase catalyzes the conversion of dihydroxyacetone phosphate into methylglyoxal and phosphate. It has surprisingly been found that deletion or functional inactivation of a microbial cell's endogenous gene encoding a methylglyoxal synthase further improves fermentative production of a oligosaccharide of interest by a microbial cell which possesses a functional variant of the native E. coli glycerol kinase GlpK, wherein the amino acid sequence of the native E. coli GlpK is set forth in SEQ ID NO: 1, wherein the functional variant possesses an amino acid residue comprising a non-ionized but polar acting side chain at the position of its amino acid sequence which corresponds to amino acid position 55 in the amino acid sequence of the native E. coli GlpK, and/or wherein said functional variant possesses an amino acid comprising an anionic side chain at the position of its amino acid sequence which corresponds to amino acid position 231 in the amino acid sequence of the native E. coli GlpK.

The genetically engineered microbial cells as described herein before can be used for the fermentative production of an oligosaccharide of interest.

Hence, also disclosed are methods for fermentative production of an oligosaccharide of interest, wherein the method comprises
providing a genetically engineered microbial cell according to the first aspect for the production of the oligosaccharide of interest, i.e. a genetically engineered microbial cell being able to intracellularly synthesize the oligosaccharide of interest when cultivated in the presence of glycerol as sole carbon source, wherein the genetically engineered microbial cell comprises a glycerol permease and a functional variant of the native E. coli glycerol kinase GlpK, wherein the amino acid sequence of the native E. coli GlpK is set forth in SEQ ID NO: 1, wherein the functional variant possesses an amino acid residue comprising a non-ionized but polar acting side chain at the position of its amino acid sequence which corresponds to amino acid position 55 in the amino acid sequence of the native E. coli GlpK, and/or wherein said functional variant possesses an amino acid comprising an anionic side chain at the position of its amino acid sequence which corresponds to amino acid position 231 in the amino acid sequence of the native E. coli GlpK, a metabolic pathway for the intracellular biosynthesis of a nucleotide-activated monosaccharide as a donor substrate of its monosaccharide moiety, and a glycosyltransferase for the transfer of the monosaccharide moiety from the nucleotide-activated monosaccharide to an acceptor molecule;
culturing the genetically engineered microbial cell in a culture medium and at conditions that are permissive for the genetically engineered microbial cell to intracellularly synthesize the oligosaccharide of interest, wherein the culture medium contains glycerol as carbon source for the genetically engineered microbial cell; and
optionally, retrieving the oligosaccharide of interest from the microbial cell and/or the culture medium.

Also disclosed are methods alleviating the toxicity of methylglyoxal to a microbial cell when the microbial cell is cultivated in the presence of glycerol as carbon source, as well as methods for decreasing the responsivity of a microbial cell to methylglyoxal toxicity during fermentative production of an oligosaccharide of interest. These methods comprise providing a genetically engineered microbial cell that synthesizes an oligosaccharide of interest intracellularly; deleting or functional inactivating the endogenous glycerol kinase gene(s) of the microbial cell; and transforming the microbial cell to contain and express a functional variant of the E. coli glycerol kinase GlpK, wherein said functional variant possesses an amino acid residue at the position which corresponds to amino acid position 55 in the amino acid sequence of E. coli GlpK as set forth in SEQ ID NO: 1, wherein said amino acid residue comprises a non-ionized but polar acting side chain, and/or an amino acid residue at the position which corresponds to amino acid position 231 in the amino acid sequence of E. coli GlpK as set forth in SEQ ID NO: 1, wherein said amino acid residue comprises an anionic side chain.

The present invention will be described with respect to particular embodiments and with reference to drawings, but the invention is not limited thereto but only by the claims. Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description and drawings provided herein, numerous specific details are set forth. However, it is understood that embodiments of the invention may be practiced without these specific details. In other instances, well-known methods, structures and techniques have not been shown in detail in order not to obscure an understanding of this description.

The invention will now be described by a detailed description of several embodiments of the invention. Other embodiments of the invention can be configured according to the knowledge of persons skilled in the art without departing from the true spirit or technical teaching of the invention, the invention being limited only by the terms of the appended claims.

### EXAMPLES

### Example 1:3-FL, ΔmgsA, GlpK (A55T)—Adaptive Laboratory Evolution of E. coli

An E. coli strain being able to produce 3-FL was subjected to an adaptive laboratory evolution experiment to investigate the impact of a cured methylglyoxal toxicity on growth and 3-FL production using glycerol as single carbon and energy source.

The E. coli strain being able to produce 3-FL had its lacZ gene deleted, expressed a heterologous alpha-1,3-fucosyltransferase gene, and had its mgsA gene deleted. All molecular biological procedures were performed as described in Sambrook, J. and Russell, D., Molecular Cloning: A laboratory Manual, 3^{rd} Edition, Cold Spring Harbor Laboratory Press, New York, 2001. To delete the mgsA gene in the E. coli strain being able to produce 3-FL, the upstream and downstream regions close to the open reading frame of mgsA were amplified by polymerase chain reaction (PCR) generating two DNA fragments. Primers 1 and 2 (Table 1) were used to generate the upstream fragment, while primers 3 and 4 (Table 1) were used to amplify the downstream region flanking mgsA. These two DNA fragments were used as templates in third PCR employing primers 1 and 4 (Table 1) to generate the DNA construct that was subsequently used to delete the mgsA gene pursant to a modified version of the method described by Datsenko, K. A. and Wanner, B. L. (One-step inactivation of chromosomal genes in Escherichia coli K-12 using PCR products, Proc. Natl. Acid. Sci 97 (2000) 6640-6645. Individual clones were analyzed by colony PCR using primers 1 and 4 (Table 1). A selected strain designated 3-FL-2 was subjected to chemical mutagenesis by exposure to N-methyl-N′-nitro-N-nitrosoguanidine, followed by two sequential fed-batch cultivations in a bioreactor. The cultivations were performed as described in WO 2018/077892 A1.

Cells obtained from the first fed-batch cultivation were used as inoculum for the second fed-batch cultivation. Cell samples were collected during the second cultivation, diluted in 0.9% (w/v) NaCl, and aliquots of 100 μL were inoculated in plates containing 2YT medium composed of 16 g/L tryptone, 10 g/L yeast extract, 5 g/L NaCl, and 18 g/L agar. After incubation for 48 h at 30° C., isolated colonies were screened for improved production of 3-FL in deep-well plates using the same batch medium as used in the fed-batch cultivations described herein before. For the precultures used in this experiment, plates were incubated for 24 h at 30° C., 800 rpm, 80% humidity (Microtron, Infors AG, Switzerland). In the main deep-well plate cultivations, the medium was supplemented with 10 mM lactose and the cultivation conditions were the same as that used for the precultures. Sample preparation and analysis by HPLC were carried out as described in WO 2018/077892 A1.

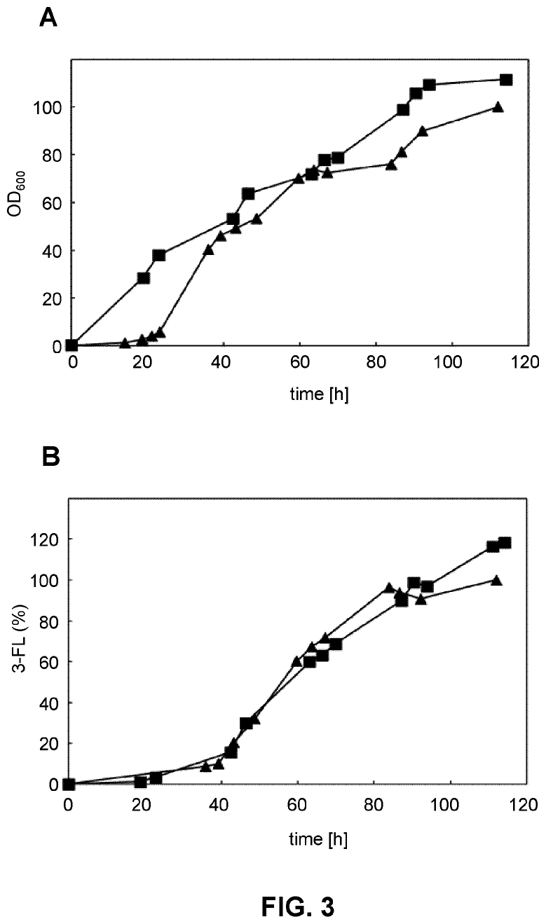

A selected E. coli isolate (3-FL-3) obtained in the adaptive laboratory evolution experiment was cultured in a bioreactor in fed-batch mode to assess its growth characteristics and its 3-FL production as compared to the growth characteristics and 3-FL production of its progenitor strain which was not subjected to the laboratory evolution experiment. The results are displayed in FIG. 3, wherein all data points are relative to the control strain.

TABLE 1
Primers

| Primer # | Nucleotide Sequence (5′→3′) | Target | SEQ ID NO: |
|---|---|---|---|
| 1 | ATGGTGTGAGTGGACGTTAAAATGC | mgsA | 3 |
| 2 | GGCGTTTGCCACCTGTGCAATATTACATAA | mgsA | 4 |
| | TGTACATCCGTAGTTAACTTTCC | | |
| 3 | GGAAAGTTAACTACGGATGTACATTATGTA | mgsA | 5 |
| | ATATTGCACAGGTGGCAAACGCCACC | | |
| 4 | GAGATTCGCGATCTGTTCAGTAAACG | mgsA | 6 |
| 5 | GTAGAACACGACCCAATGGAAATCTGGAC | glpK | 7 |
| | GACCCAAAGCTCCACGCTGGTAGAAGTGC | | |
| | TGGCGAAAGCCGATATCAGTTCCGATCAAA | | |
| | TT | | |
| 6 | CCCAATGGAAATCTGGGCC | glpK | 8 |
| 7 | CGACCCAATGGAAATCTGGACG | glpK | 9 |
| 8 | GCCAATGTTAGTCTGACCGTATACTTCGG | glpK | 10 |
| 9 | GCTTCGCTGTAATATGACTACGGGAC | glpK | 11 |
| 10 | GATGAGCTGGGTGGAACGGCATCAACCGT | mgsA | 12 |
| | TACTGGAACAACACGTACTGTAACGCAACA | | |
| | GGCACTACCGGTAACTTAATTTCCCGCGC | | |
| 11 | CGTTACTGGAACAACACGTACTGTAAC | mgsA | 13 |
| 12 | CGTTACTGGAACAACACGTACTGTAT | mgsA | 14 |
| 13 | CCACCGGTTGAGGATTTCCCG | mgsA | 15 |
| 14 | CCTGTGCGCAGGCTTTTTCG | mgsA | 16 |
| 15 | GGCGATCCCGGAGATTGGAATACGCGTGC | glpK | 17 |
| | CGCCTTTGCCATCAATGTTAGTCTGACCGT | | |
| | ATACTTCGGAAGAACGACGCACTTCTGG | | |
| 16 | CGAAGTATACGGTCAGACTAACATTGAT | glpK | 18 |
| 17 | AGTATACGGTCAGACTAACATTGGC | glpK | 19 |
| 18 | GCGTGCAGACGGATACCAGAG | glpK | 20 |
| 19 | GCTGTTTGGTACGGTTGATACGTGG | glpK | 21 |

The selected E. coli isolate 3-FL-3 (▪) exhibited a 44% reduction in time for concluding the batch phase (FIG. 3A) as compared to progenitor strain (▴) when analyzing the optical density of the culture medium at a wavelength of 600 nm (OD₆₀₀). In addition, the selected E. coli isolate 3-FL-3 (▪) displayed an increase of 3-FL in the culture supernatant of about 18% after nearly 100 hours of culturing as compared to the amount of 3-FL found in the culture supernatant of its progenitor strain (▴) (FIG. 3B).

The selected E. coli isolate 3-FL-3 showing faster growth and increased 3-FL production as compared to its progenitor strain was found to contain a mutated glpK gene encoding a variant of GlpK wherein the alanine residue at position 55 is replaced with a threonine residue. This variant of E. coli GlpK is designated GlpK (A55T).

### Example 2: Production of 2′-FL or LNT by GlpK Mutant E. coli Strains

To investigate the effect of GlpK (A55T) on production of other HMOs than 3-FL, E. coli strains able to produce 2′-FL or LNT were genetically engineered in that their endogenous glpK gene was replaced by a variant that encodes E. coli (K12) GlpK (A55T). All molecular biological procedures used for the modifying the endogenous glpK gene into the mutant glpK gene in both the 2′-FL-producing and the LNT-producing E. coli strains were performed by methods described in Sambrook, J. and Russell, D., Molecular Cloning: A laboratory Manual, 3^{rd} Edition, Cold Spring Harbor Laboratory Press, New York, 2001. For altering the nucleotide sequence of the native glpK gene primer 5 (Table 1) was used for the recombination event.

To identify clones harboring the mutant glpK gene, colony PCRs were carried out using a combination of primers 6 and 8 (Table 1) for confirming presence of the wild-type glpK gene, while a combination of primers 7 and 8 (Table 1) was used for confirming presence of the mutated version of glpK. Bacterial clones identified to bear the mutated version of the glpK gene were subjected to nucleotide sequence analysis using primers 8 and 9 (Table 1) for confirming that they bear the variant of the glpK gene which encodes GlpK (A55T). The thus constructed 2′-FL producing E. coli strain was named E. coli 2′-FL-2 (Table 2). The thus constructed LNT-producing E. coli strain was based on a metabolically engineered E. coli BL21 (DE3) similar to the E. coli LNT-1 strain (Table 2) and was named E. coli LNT-2 (Table 2). Both E. coli strains, 2′-FL2 and LNT-2, were cultivated in fed-batch processes to compare the growth characteristics and HMO production profiles. The fed-batch cultivations and HPLC analyses were carried out in similar conditions as described in WO 2018/077892 A1.

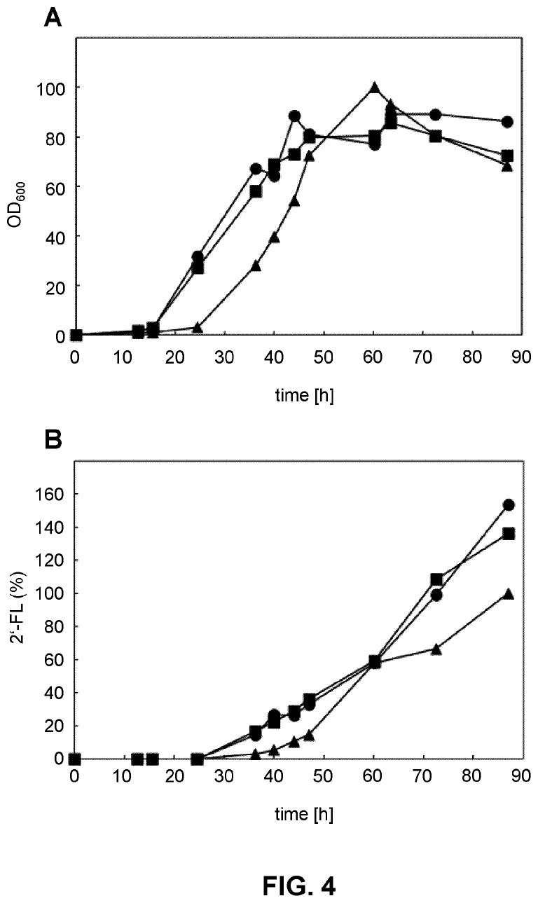

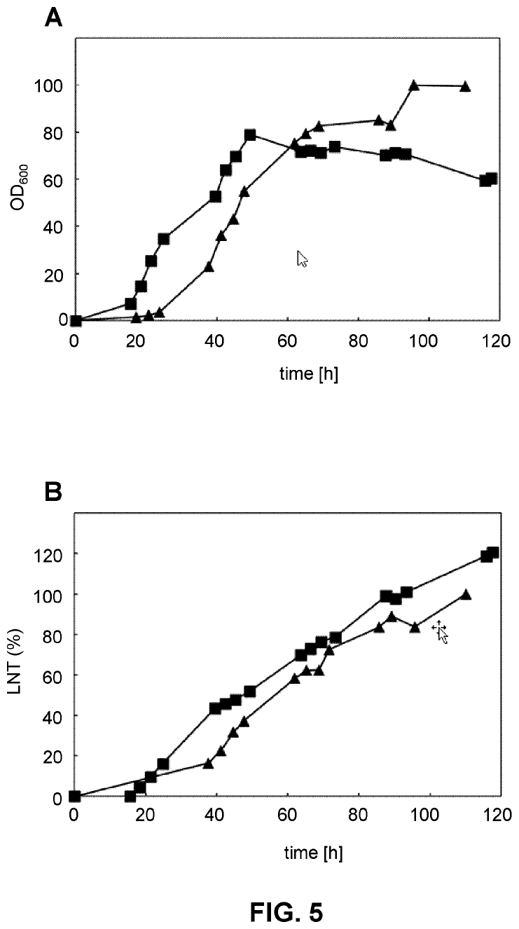

Despite the presence of the endogenous mgsA gene in E. coli 2′-FL-2 and E. coli LNT-2 strains, they showed improved growth (FIG. 4A and FIG. 5A) and HMO production (FIG. 4B and FIG. 5B) as compared to the respective control strains (▴). For the E. coli 2′-FL-2 strain (Table 2) (▪), FIG. 4A displays the reduction in time required for concluding the batch phase and FIG. 4B illustrates the increase of the 2′-FL titer. Reduction in time for concluding the batch phase was about 35% while the 2′-FL titer was increased by about 36%. In FIG. 5, the 41% reduction in time for concluding the batch phase by the E. coli LNT-2 strain (Table 2) (▪) as compared to its control strain (A) is shown in FIG. 5A. Further, the LNT-2 titer in the culture supernatant of E. coli 2′-FL-2 (▴) was increased by about 14% (see FIG. 5B).

TABLE 2
E. coli strains.

| Name | Relevant genotype |
|---|---|
| 3-FL-1 | BL21 (DE3) ΔlacZ ΔwcaJ ΔfucIK |
| | manB manC gmd wcaG fucT setA |
| 3-FL-2 | 3-FL-1 ΔmgsA |
| 3-FL-3 | 3-FL-2 glpK(G163A) |
| 2′-FL-1 | BL21 (DE3) ΔlacZ ΔaraA galETKM ΔwzxC |
| | ΔwcaJ ΔfucIK manB manC gmd wcaG lacY |
| | yberc0001_9420 wbgL ΔpfkA fkp fbaB-fbpase |
| 2′-FL-2 | E. coli 2′-FL-1 glpK(G163A, C165G) |
| 2′-FL-3 | E. coli 2′-FL-2 ΔmgsA |
| 2′-FL-4 | E. coli 2′-FL-1 glpK(G692A, C693T) |
| LNT-1 | JBT-LNT |
| LNT-2 | E. coli LNT-1 glpK(G163A, C165G) |
| LNT-3 | E. coli LNT-2 ΔmgsA |

### Example 3 Overcoming Growth Inhibition Through mgsA Inactivation in 2′-FL and LNT Production Strains Harboring the GlpK (A55T)

To make sure that methylglyoxal toxicity does not become an issue during HMO production using E. coli strains bearing GlpK (A55T), the growth kinetics of different E. coli strains bearing GlpK (A55T) for 2′-FL or LNT production were recorded at different production conditions. Growth kinetics were compared between E. coli strains bearing a native mgsA gene and descendent strains having their mgsA gene deleted.

Sudden exposure of E. coli strains harboring a feedback resistant variant of GlpK to glycerol may lead to an inhibition of cell growth. During fermentative production of HMOs, usually the bacterial preculture obtained in seed fermenters is used for inoculating the main fermenter while the bacterial cells are still in exponential growth phase. However, it may occur that the bacterial cell culture in the seed fermenter has reached stationary growth phase before the main fermenter can be inoculated. This would lead to an exposure of bacterial cells to a high glycerol concentration in the main fermenter when being inoculated which in turn would lead to methylglyoxal toxicity.

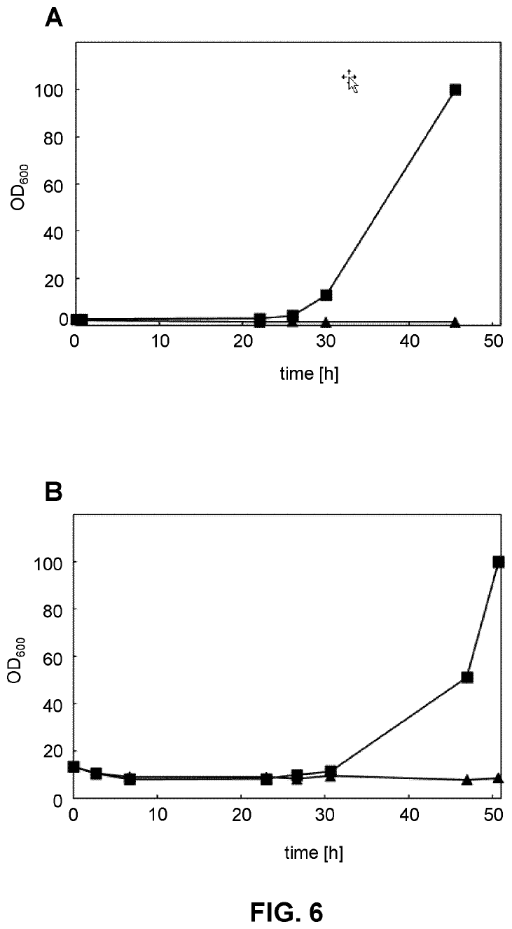

To investigate whether such scenario could lead to growth problems and affect production of HMOs, E. coli strains bearing GlpK (A55T) and being able to synthesize 2′-FL or LNT were tested using the following approach. A fed-batch preculture was prepared to obtain an inoculum to be used for HMO production in fed-batch mode of operation, as described in example 2. As can be seen in FIG. 6, both E. coli strains, 2′-FL-2 (▴) (Table 2; FIG. 6A) and LNT-2 (▴) (Table 2; FIG. 6B) were not able to grow in the main fermenter when their pre-cultures originated from a fed-batch cultivation.

Subsequently, the experiment was repeated using descendent 2′-FL-producing or LNT-producing E. coli strains that additionally had their mgsA gene deleted. For inactivating the mgsA gene in E. coli 2′-FL-2 and E. coli LNT-2 strains, the same single stranded DNA based method described in example 2 was used. Primer 10 (Table 1) was used to functionally inactivate the mgsA gene by inserting a stop codon in position 43 and a frameshift. A combination of primers 11 and 13 (Table 1) was used to confirm functional inactivation of the mgsA gene. A combination of primers 12 and 13 (Table 1) was used to confirm presence of the endogenous mgsA gene. Individual bacterial clones were selected after colony PCR verifying functional inactivation of mgsA, and they were further characterized by nucleotide sequence analysis using primers 13 and 14 (Table 1).

In the case of the E. coli ΔmgsA variant, designated 2′-FL-3, of the E. coli strain 2′-FL-2 for producing 2′-FL (Table 2; FIG. 6A), and the E. coli ΔmgsA variant, designated LNT-3, of E. coli strain LNT-2 (Table 2; FIG. 6B), cell growth could be observed for both strains (▪) when challenged with glycerol upon inoculation from stationary growth phase.

These results demonstrate the importance of the ΔmgsA genotype as a preventive measure and/or to further increase robustness of bacterial HMO production strains to be able to cope with plant operation variability.

### Example 4. Comparing 2′-FL Production by E. coli Bearing GlpK (A55T) or GlpK (G231D)

The results shown in examples 1 and 2 led us to investigate whether other variants of GlpK could also improve HMO production. To test this hypothesis, the 2′-FL productivity of E. coli strain 2′-FL-2 (Table 2) was compared to that of E. coli strain 2′-FL-4 (Table 2) which contains a glpK gene encoding the previously reported feedback resistant version GlpK (G231D) (Honisch, C. et al. (2004) Genome Res. 14:2495-2502). The E. coli strain 2′-FL-4 (Table 2) was constructed by replacing its native glpK gene with the glpK (G692A, C693T) gene in the same E. coli precursor strain that was used for constructing E. coli strain 2′-FL-2 (Table 2). To improve the efficiency of the genetic engineering procedure employed, the glpK gene mutations G692A and C693T were used in combination instead of only the literature described mutation G692A. Both the nucleotide mutation G692A or the G692A mutation in combination with C693T code for the target GlpK enzyme that has the amino acid substitution G231D. Construction of the E. coli 2′-FL-4 strain (Table 2) followed the same methodology used in example 2 for creation of the E. coli 2′-FL-2 strain (Table 2). The primer 15 (Table 1) was used for the glpK gene modification. For clone selection, either the primer combination 16 and 18 (Table 1) or the pair 17 and 18 (Table 1) were employed for the identification of the mutant glpK variant and the native glpK gene, respectively. To confirm the desired modification, the glpK gene range was sequenced using the primers 18 and 19 (Table 1). Similar to the E. coli 2′-FL-2 strain (Table 2), the E. coli 2′-FL-4 (Table 2) was also cultivated in a fed-batch for 2′-FL production using the same cultivations conditions and analytical procedures described in example 2. The G231D mutation in the GlpK enzyme of strain E. coli 2′-FL-4 (•) (Table 2) allowed an even higher positive effect on 2′-FL production (FIG. 4B). The 2′-FL titer by E. coli 2′-FL-4 (•) increased by 53% as compared to the 2′-FL titer by the control strain (▴). In addition, a reduction in batch time of 34% compared to the control strain (▴) was reached.

1. A genetically engineered microbial cell for intracellular biosynthesis of an oligosaccharide of interest, wherein the genetically engineered microbial cell synthesizes the oligosaccharide of interest intracellularly when cultivated in the presence of glycerol as sole carbon and energy source, wherein the genetically engineered microbial cell comprises
a glycerol permease for internalization of exogenous glycerol;
a glycerol kinase, wherein said glycerol kinase is a functional variant of the E. coli glycerol kinase GlpK, wherein said functional variant possesses an amino acid residue at the position which corresponds to amino acid position 55 in the amino acid sequence of E. coli GlpK as set forth in SEQ ID NO: 1, wherein said amino acid residue comprises a non-ionized but polar acting side chain, and/or an amino acid residue at the position which corresponds to amino acid position 231 in the amino acid sequence of E. coli GlpK as set forth in SEQ ID NO: 1, wherein said anionic amino acid residue comprises an anionic side chain;
a metabolic pathway for the intracellular biosynthesis of a nucleotide-activated monosaccharide as a donor substrate of the monosaccharide moiety; and
a glycosyltransferase for the transfer of the monosaccharide moiety from the nucleotide-activated monosaccharide to an acceptor substrate.

2. The genetically engineered microbial cell according to claim 1, wherein the amino acid residue with a non-ionized but polar acting side chain is selected from the group consisting of L-serine, L-threonine, L-cysteine, L-methionine, L-asparagine and L-glutamine.

3. The genetically engineered bacterial cell according to claim 1, wherein the anionic amino acid residue is selected from the group consisting of L-aspartate and L-glutamate.

4. The genetically engineered microbial cell according to claim 1, wherein the functional variant has a sequence identity to E. coli (K12) GlpK of at least 80%.

5. The genetically engineered microbial cell according to claim 1, wherein the genetically engineered microbial cell has been genetically engineered to contain and express a recombinant gene which encodes the functional variant of the E. coli GlpK.

6. The genetically engineered microbial cell according to claim 5, wherein
(i) the endogenous glycerol kinase gene(s) of the microbial cell has/have been deleted or functionally inactivated, and wherein the microbial cell has been transformed to contain and express a recombinant gene which encodes the functional variant of the E. coli GlpK; or
(ii) the endogenous glycerol kinase gene(s) of the microbial cell has/have been edited to contain and express a recombinant gene which encodes the functional variant of the E. coli GlpK.

7. The genetically engineered microbial cell according to claim 1, wherein the endogenous methylglyoxal synthase gene(s) has/have been deleted or functionally inactivated.

8. The genetically engineered microbial cell according to claim 1, wherein the oligosaccharide of interest is an oligosaccharide selected from the group consisting of human milk oligosaccharides.

9-10. (canceled)

11. A method for the fermentative production of an oligosaccharide of interest, the method comprises
providing a genetically engineered microbial cell according to claim 1 for intracellular biosynthesis of the oligosaccharide of interest;
culturing the genetically engineered microbial cell in a culture broth which contains glycerol as carbon- and energy source for the genetically engineered microbial cell; and
retrieving the oligosaccharide of interest from the microbial cell and/or the culture broth.

12. The method according to claim 11, wherein the glycerol is the sole carbon and energy source being exogenously added to the culture broth.

13. The method according to claim 11, wherein the oligosaccharide of interest is an oligosaccharide selected from the group consisting of human milk oligosaccharides.

14. A method for alleviating methyl glyoxal toxicity of a microbial cell, wherein the method comprises:
providing a genetically engineered microbial cell for the fermentative production of an oligosaccharide of interest;
deleting or functionally inactivating the endogenous glycerol kinase gene(s) of the microbial cell;
transforming the microbial cell to contain and express a gene which encodes a functional variant of the E. coli (K12) glycerol kinase GlpK, wherein said functional variant possesses an amino acid residue at the position which corresponds to amino acid position 55 in amino acid sequence of E. coli GlpK as set forth in SEQ ID NO: 1, wherein said amino acid residue comprises a non-ionized but polar acting side chain, and/or an amino acid residue at the position which corresponds to amino acid position 231 in the amino acid sequence of E. coli GlpK as set forth in SEQ ID NO: 1, wherein said anionic amino acid residue comprises an anionic side chain.

15. A method for decreasing the responsivity of a microbial cell to methylglyoxal toxicity during fermentative production of an oligosaccharide of interest, wherein the method comprises:
providing a genetically engineered microbial cell for the fermentative production of an oligosaccharide of interest;
deleting or functionally inactivating the endogenous glycerol kinase gene(s) of the microbial cell;
transforming the microbial cell to contain and express a gene which encodes a functional variant of the E. coli glycerol kinase GlpK, wherein said functional variant possesses an amino acid residue at the position which corresponds to amino acid position 55 in amino acid sequence of E. coli GlpK as set forth in SEQ ID NO: 1, wherein said amino acid residue comprises a non-ionized but polar acting side chain, and/or an amino acid residue at the position which corresponds to amino acid position 231 in the amino acid sequence of E. coli GlpK as set forth in SEQ ID NO: 1, wherein said anionic amino acid residue comprises an anionic side chain.
